(19) United States
(12) Patent Application Publication
(10) Pub. No.: US 2008/0221422 A1
(43) Pub. Date: Sep. 11, 2008

(54) SENSOR MEASUREMENT SYSTEM HAVING A MODULAR ELECTRODE ARRAY AND METHOD THEREFOR

(75) Inventor: Borje Rantala, Helsinki (FI)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/683,514

(22) Filed: Mar. 8, 2007

(51) Int. Cl. A61B 5/04

(52) U.S. Cl. 600/372

(57) ABSTRACT

A method for implementing a sensor measurement system is disclosed herein. The method includes providing a plurality of generally identical modular sensor arrays each having a single array connector, applying two of the plurality of modular sensor arrays to a patient such that the two modular sensor arrays can receive biopotential signals from the patient, and coupling the array connector of each of the two modular sensor arrays with a monitor such that the two modular sensor arrays can transmit the biopotential signals to the monitor and the monitor can convey information pertaining to the biopotential signals in a selectable form. A corresponding sensor measurement system is also provided.

### FIELD OF THE INVENTION

This disclosure relates generally to a sensor measurement system having a modular electrode array, and a method for implementing the sensor measurement system.

### BACKGROUND OF THE INVENTION

Electroencephalography (EEG) is a well established method for assessing brain function by picking up weak biosignals generated in the brain. To obtain the biosignals, multiple electrodes are placed on a patient's head in accordance with a recognized protocol. The electrodes are generally individually coupled to a monitor with a series of wires. The monitor is configured to display information pertaining to the biosignals in a selectable form.

One protocol for placing the electrodes involves the placement of a relatively large number of electrodes (e.g., 20 electrodes) at predetermined locations on the patient's scalp. A problem with this protocol is that it is inconvenient to attach such a large number of electrodes. More precisely, applying each electrode to the patient takes time and skill, requires skin preparation, and is especially difficult for patients with thick hair. Additionally, individually forming the electrical connections between each electrode and a monitor is a time consuming process that is subject to human error. As an example, unless each electrode is properly connected to an appropriate monitor input, the information conveyed by the monitor may be imprecise or unclear.

Another protocol for placing electrodes involves the placement of a relatively small number of electrodes (e.g., 3 electrodes) that are generally positioned on the patient's forehead. It should be appreciated that systems having fewer electrodes are easier to apply to the patient, easier to connect to the monitor, and are less prone to human error when being connected to the monitor. One problem with systems incorporating a relatively small number of electrodes is that they generally convey less information than comparable systems having additional electrodes.

### BRIEF DESCRIPTION OF THE INVENTION

The above-mentioned shortcomings, disadvantages and problems are addressed herein which will be understood by reading and understanding the following specification.

In an embodiment, a method for implementing a sensor measurement system includes providing a plurality of generally identical modular sensor arrays each having a single array connector, applying two of the plurality of modular sensor arrays to a patient such that the two modular sensor arrays can receive biopotential signals from the patient, and coupling the array connector of each of the two modular sensor arrays with a monitor such that the two modular sensor arrays can transmit the biopotential signals to the monitor and the monitor can convey information pertaining to the biopotential signals in a selectable form.

In another embodiment, a method for implementing a sensor measurement system includes providing a first modular sensor array having a first plurality of electrodes and a first array connector, and providing a second modular sensor array generally identical to the first modular sensor array. The second modular sensor array has a second plurality of electrodes and a second array connector. The method also includes applying the first plurality of electrodes and the second plurality of electrodes to a patient such that the first modular sensor array and the second modular sensor array can receive biopotential signals from the patient. The method also includes coupling the first array connector and the second array connector with a monitor such that the first modular sensor array and the second modular sensor array can transmit the biopotential signals to the monitor and the monitor can convey information pertaining to the biopotential signals in a selectable form.

In another embodiment, a sensor measurement system includes a monitor, and a first modular sensor array operatively connected to the monitor. The first modular sensor array has a first plurality of electrodes. The sensor measurement system also includes a second modular sensor array operatively connected to the monitor. The second modular sensor array is generally identical to the first modular sensor array. The second modular sensor array has a second plurality of electrodes. The first plurality of electrodes and the second plurality of electrodes are adapted to transmit biopotential signals from a patient to the monitor so that the monitor can convey information pertaining to the biopotential signals in a selectable form.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a modular sensor array in accordance with an embodiment;

FIG. 2 is a schematic diagram illustrating a sensor measurement system in accordance with an embodiment;

FIG. 3 is a schematic diagram illustrating a sensor measurement system in accordance with an embodiment;

FIG. 4 is a schematic diagram illustrating the modular sensor array of FIG. 1 attached to a patient; and

FIG. 5 is a schematic diagram illustrating two of the modular sensor arrays of FIG. 1 attached to a patient.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

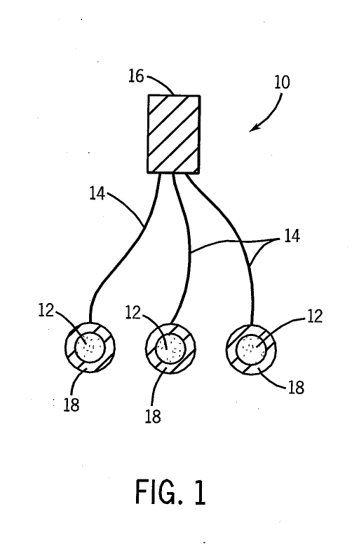

Referring to FIG. 1, a modular sensor array 10 is shown in accordance with an embodiment. The modular sensor array 10 is referred to as being “modular” because it defines a standardized unit that may be implemented individually or in combination with other generally identical devices. The modular sensor array 10 is referred to as being an “array” because it comprises multiple sensors (e.g., the electrodes 12).

The modular sensor array 10 is depicted as including three electrodes 12, however it should be appreciate that alternate embodiments may include other types of sensors and/or additional sensors. Each electrode 12 is operatively connected to a conductor 14. The conductors 14 are each configured to transmit biopotential signals from a respective electrode 12. The electrodes 12 and conductors 14 may be formed of a conductive material suitable for receiving and transmitting signals such as, for example, metallic foil or wire, vapor deposited or printed metallic layers, etc. The conductors 14 each include a first end that is connected to a respective electrode 12, and a second end that is connected to an array connector 16. An adhesive material 18 is generally disposed around the periphery of each electrode 12 in order to secure the electrodes 12 to a patient.

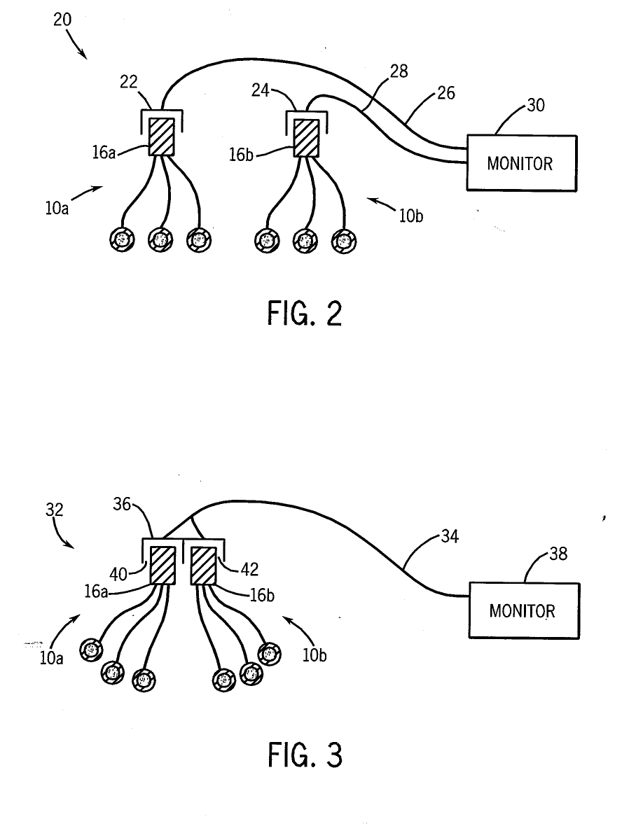

Referring to FIG. 2, a sensor measurement system 20 is shown. The sensor measurement system 20 will hereinafter be described in accordance with an embodiment having two modular sensor arrays, however, it should be appreciated that alternate embodiments may incorporate additional or fewer modular sensor arrays. The two modular sensor arrays shown in FIG. 2 are generally identical to the modular sensor array 10 (shown in FIG. 1) and will therefore be identified using base reference numbers consistent with those of FIG. 1 and a unique alphanumeric suffix.

The sensor measurement system 20 includes a first modular sensor array 10a and a generally identical second modular sensor array 10b. An array connector 16a of the first modular sensor array 10a is coupled with a first monitor connector 22, and an array connector 16b of the second modular sensor array 10b is coupled with a second monitor connector 24. According to the illustrated embodiment, the array connectors 16a, 16b are male type connectors that are insertably coupled with female type monitor connectors 22, 24. It should, however, be appreciated that the connectors 16a, 16b, 22 and 24 may include any known device configured to establish an electrical connection. The first and second monitor connectors 22, 24 are respectively coupled with a first and second monitor cable 26, 28, and the first and second monitor cables 26, 28 are coupled with a monitor 30. Accordingly, the first and second modular sensor arrays 10a, 10b transmit boipotential signals from a patient through the cables 26, 28, respectively, and to the monitor 30 which is configured to display electroencephalogram (EEG) data in a desired form.

According to an embodiment shown in FIG. 3, a sensor measurement system 32 includes a single monitor cable 34 having a dual input monitor connector 36 configured to couple the modular sensor arrays 10a, 10b with a monitor 38. The dual input monitor connector 36 includes a first input port 40 adapted to accommodate the array connector 16a, and a second input port 42 adapted to accommodate the array connector 16b. In all other respects, the sensor measurement system 32 is similar to the sensor measurement system 20 (shown in FIG. 2).

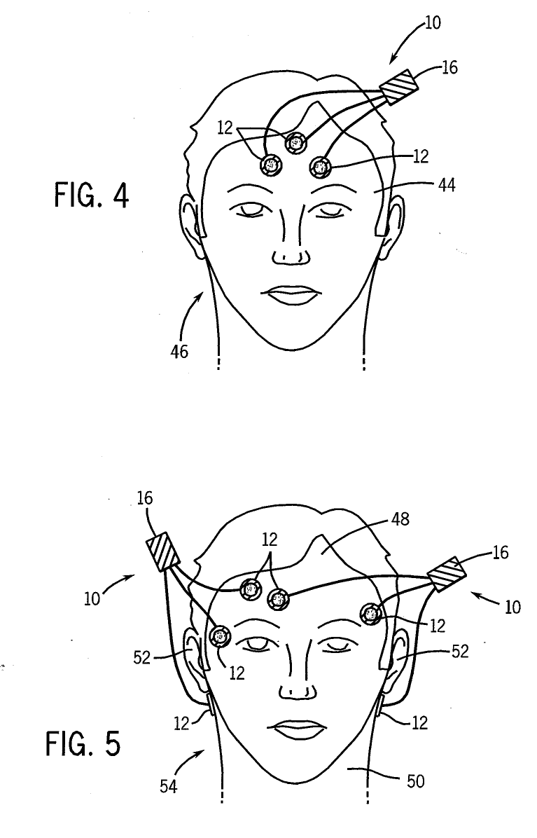

Referring to FIGS. 4 and 5, it can be seen that a single modular sensor array 10 may be implemented for applications requiring less detailed information, and multiple modular sensor arrays 10 can be implemented for applications requiring more detailed information. Therefore, the modular sensor array 10 provides a single device that is flexible enough to accommodate a variety of different applications in a convenient and efficient manner. As an example, the modular sensor arrays 10 can be sterilized and pre-packaged, and thereafter stored in a common location and identified by a common part number. Accordingly, a user can conveniently obtain one or more modular sensor arrays 10 from a single location whereas it may have previously been necessary to acquire a plurality of different components (e.g., electrodes, conductors, connectors, adhesive) from a plurality of different locations. Additionally, by using a common part number, it is less burdensome for a hospital to inventory and track the modular sensor arrays 10.

Referring to FIG. 4, an electrode placement for a single modular sensor array embodiment is illustrated. When only one modular sensor array 10 is implemented, the electrodes 12 may be attached to the forehead 44 of a patient 46 in the manner shown. The array connector 16 can then be connected to one of the monitor connectors 22, 24 (shown in FIG. 2) or inserted into one of the ports 40, 42 of a dual input monitor connector 36 (shown in FIG. 3) to couple the modular sensor array 10 with a monitor.

Referring to FIG. 5, an electrode placement for a dual modular sensor array embodiment is illustrated. The electrode placement depicted in FIG. 5 is sometimes referred to as a “sub-hairline montage” because the electrodes 12 are all applied below the patient's hairline. Advantageously, the sub-hairline montage requires minimal skin preparation, it obviates the need for hair removal, and it prevents the patient's hair from interfering with biopotential signal reception. It should, however, be appreciated that the, electrode placement shown in FIG. 5 and described hereinafter is provided for illustrative purposes, and that alternate electrode placements may be envisioned.

When two modular sensor arrays 10 are implemented, four of the electrodes 12 can be attached to the patient's forehead 48 in a spaced apart manner and the remaining two electrodes 12 can be attached to opposing sides of the patient's neck 50 below and in close proximity to the patient's ears 52. Advantageously, the implementation of two generally identical modular sensor arrays 10 on a single patient 54 can provide more detailed information than a system incorporating only three electrodes. Additionally, a sensor measurement system incorporating the modular sensor arrays 10 is easily applied to a patient (ease of application), and less prone to application error (correctness of application). The ease of application and the correctness of application of the modular sensor arrays 10 will hereinafter be described in detail.

The modular sensor array's ease of application can be illustrated by the following example and with reference to FIG. 5. A user can obtain two pre-packaged modular sensor arrays 10 from a common location. Thereafter, each pre-packaged modular sensor array 10 can be individually opened and applied to the patient 54 in the manner described hereinabove. The array connectors 16 of the two pre-packaged modular sensor arrays 10 can then be coupled with a complementary monitor connection device such as the monitor connectors 22, 24 (shown in FIG. 2) or the dual input monitor connector 36 (shown in FIG. 3). This example shows that a system incorporating the modular sensor arrays 10 may be more easily applied than a conventional system wherein a user obtains a plurality of different components (e.g., electrodes, conductors, connectors, adhesive) from a plurality of different locations, applies six electrodes to a patient, and then individually couples each of the six electrodes with a monitor.

The modular sensor array's correctness of application is related in part to the fact that each modular sensor array 10 can be coupled to a monitor using a single array connector 16. Therefore, a system incorporating two modular sensor arrays 10 only requires the formation of two electrical connections in order to couple all six electrodes 12 with a monitor. It should be appreciated that a user is less likely to improperly form one of the two electrical connections required for a system incorporating the modular sensor arrays 10 than to improperly form one of the six electrical connections of a conventional system. In other words, the likelihood that a user will improperly form an electrical connection is directly proportional to the number of electrical connections the user must form.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

I claim:

1. A method for implementing a sensor measurement system comprising:
providing a plurality of generally identical modular sensor arrays each having a single array connector;
applying two of the plurality of modular sensor arrays to a patient such that the two modular sensor arrays can receive biopotential signals from the patient; and
coupling the array connector of each of the two modular sensor arrays with a monitor such that the two modular sensor arrays can transmit the biopotential signals to the monitor and the monitor can convey information pertaining to the biopotential signals in a selectable form.

2. The method of claim 1, wherein said providing a plurality of generally identical modular sensor arrays includes providing a plurality of generally identical modular sensor arrays each having at least four electrodes.

3. The method of claim 1, wherein said providing a plurality of generally identical modular sensor arrays includes providing a plurality of generally identical modular sensor arrays each having three electrodes.

4. The method of claim 1, wherein said applying two of the plurality of modular sensor arrays to a patient includes implementing an adhesive material.

5. The method of claim 1, wherein said coupling the array connector of each of the two modular sensor arrays with a monitor includes implementing two monitor connectors each operatively connected to an individual monitor cable.

6. The method of claim 1, wherein said coupling the array connector of each of the two modular sensor arrays with a monitor includes implementing a dual input monitor connector operatively connected to a monitor cable.

7. A method for implementing a sensor measurement system comprising:
providing a first modular sensor array having a first plurality of electrodes and a first array connector;
providing a second modular sensor array generally identical to the first modular sensor array, said second modular sensor array having a second plurality of electrodes and a second array connector;
applying the first plurality of electrodes and the second plurality of electrodes to a patient such that the first modular sensor array and the second modular sensor array can receive biopotential signals from the patient; and
coupling the first array connector and the second array connector with a monitor such that the first modular sensor array and the second modular sensor array can transmit the biopotential signals to the monitor and the monitor can convey information pertaining to the biopotential signals in a selectable form.

8. The method of claim 7, wherein said providing a first modular sensor array having a first plurality of electrodes includes providing a first modular sensor array having at least four electrodes.

9. The method of claim 7, wherein said providing a first modular sensor array having a first plurality of electrodes includes providing a first modular sensor array having three electrodes.

10. The method of claim 7, wherein said applying the first plurality of electrodes and the second plurality of electrodes to a patient includes implementing an adhesive material.

11. The method of claim 7, wherein said coupling the first array connector and the second array connector with a monitor includes implementing two monitor connectors each operatively connected to an individual monitor cable.

12. The method of claim 7, wherein said coupling the first array connector and the second array connector with a monitor includes implementing a dual input monitor connector operatively connected to a monitor cable.

13. A sensor measurement system comprising:
a monitor;
a first modular sensor array operatively connected to the monitor, said first modular sensor array having a first plurality of electrodes; and
a second modular sensor array operatively connected to the monitor, said second modular sensor array generally identical to the first modular sensor array, said second modular sensor array having a second plurality of electrodes;
wherein the first plurality of electrodes and the second plurality of electrodes are adapted to transmit biopotential signals from a patient to the monitor so that the monitor can convey information pertaining to the biopotential signals in a selectable form.

14. The sensor measurement system of claim 13, wherein the first plurality of electrodes includes three electrodes, and the second plurality of electrodes includes three electrodes.

15. The sensor measurement system of claim 13, wherein the first modular sensor array includes a first array connector, and the second modular sensor array includes a second array connector.

16. The sensor measurement system of claim 13, wherein the first modular sensor array includes a first plurality of conductors adapted to couple the first plurality of electrodes with the first array connector, and wherein the second modular sensor array includes a second plurality of conductors adapted to couple the second plurality of electrodes with the second array connector.

17. The sensor measurement system of claim 13, wherein the monitor includes a first monitor connector adapted for attachment with the first array connector, and a second monitor connector adapted for attachment with the second array connector.

18. The sensor measurement system of claim 13, wherein the monitor includes a dual input monitor connector for attachment with the first array connector and the second array connector.
